(19) United States
(12) Patent Application Publication
(10) Pub. No.: US 2014/0015928 A1
(43) Pub. Date: Jan. 16, 2014

(54) DEVICE FOR RECORDING IMAGES OF THREE-DIMENSIONAL OBJECTS

(71) Applicants: Horst Koinig, Klagenfurt (AT); Jürgen Jesenko, Maria Rain (AT)

(72) Inventors: Horst Koinig, Klagenfurt (AT); Jürgen Jesenko, Maria Rain (AT)

(73) Assignee: a.tron 3d GmbH, Klagenfurt am Worthersee (AT)

(21) Appl. No.: 14/005,840

(22) PCT Filed: Mar. 12, 2012
(86) PCT No.: PCT/AT12/00061

§ 371 Date: Oct. 2, 2013

(30) Foreign Application Priority Data
Mar. 18, 2011 (AT) A 388/2011

(51) Int. Cl. A61B 1/00

(52) U.S. Cl. CPC A61B 1/00193
USPC 348/45

(57) ABSTRACT

A device for the three-dimensional recording of objects (10), in particular teeth, includes a recording region (2), in which at least one mirror (26, 27, 28) is arranged in order to deflect a light or projection beam (23) and/or an image reflected by the object (10), and a gripping region (3). A camera (32) and/or a projector (14) is/are arranged in the recording region (2), and the recording region (2) is tilted against the projection direction at an angle (a) between 10° and 40° relative to the gripping region (3).

The invention relates to a device for recording images of three-dimensional objects, in particular teeth, with an imaging area, in which at least one minor is arranged in order to deflect a light or projection beam and/or an image that is reflected by the object, and with a gripping area.

Such devices are used in particular in the area of the three-dimensional imaging of teeth. In this case, the application extends to the recording of digital tooth and jaw impressions, the support in diagnosis, the monitoring of dental treatments as well as the reliable monitoring of inserted implants. In addition to other uses in the field of medical and industrial technology, for example in the field of endoscopy, objects that are difficult to access can generally be measured stereometrically.

From the state of the art, for example AT 508 563 B, a device and a method for measuring objects, such as teeth, that yield three-dimensional images are already known. This device and method, but also other known devices and methods, can be used in this invention to produce three-dimensional images.

A significant drawback of known devices is that the imaging devices (handheld scanners), which penetrate the objects to be imaged, in most cases are unwieldy, bulky and geometrically not shaped for efficient guiding into the patient's mouth.

The object of the invention is therefore to make available a device that is easy to handle and that makes possible an efficient measuring in the mouth.

This object is achieved in a device of the above-mentioned type in that a camera and/or a projector is/are arranged in the imaging area and in that the imaging area is tilted against the projection direction by an angle of between 10° and 40° relative to the gripping area.

Owing to the fact that the imaging area is curved against the projection direction by an angle of between 10° and 40° relative to the gripping area, the view of the individual using the device of the object to be imaged is no longer hampered by the handpiece or the hand that holds the handpiece, thus facilitating a more efficient imaging of objects even under cramped conditions.

Preferred embodiments of the invention are subjects of the dependent claims.

Additional features and advantages of the invention will emerge from the subsequent description of preferred embodiments of the invention with reference to the attached drawings.

Here:

FIG. 1 shows an embodiment of a handpiece for the invention from the side,

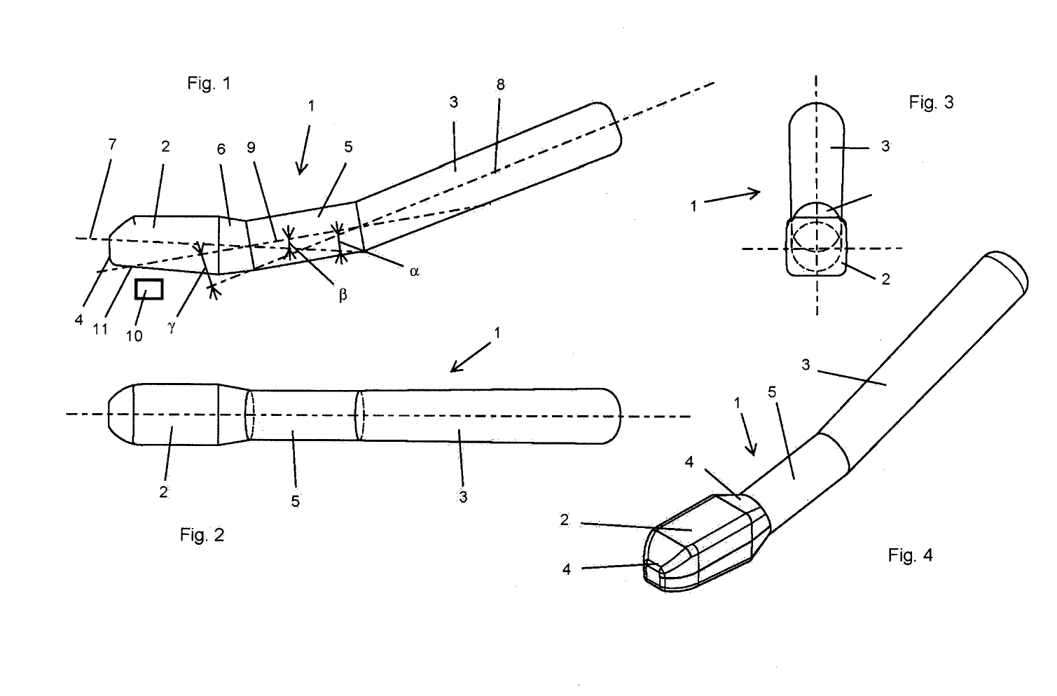

FIG. 2 shows the handpiece of FIG. 1 in top view,

FIG. 3 shows the handpiece of FIG. 1 from the front,

FIG. 4 shows the handpiece of FIGS. 1 to 3 in an oblique cutaway view,

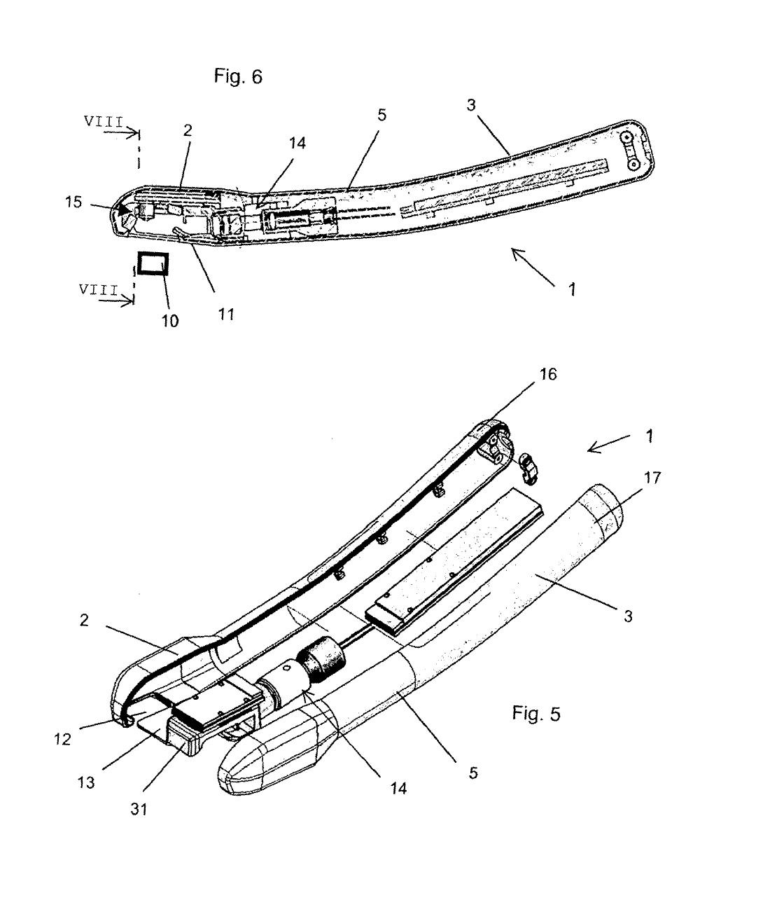

FIG. 5 shows a partial exploded view of an embodiment of the invention,

FIG. 6 shows a longitudinal section through the embodiment of FIG. 5,

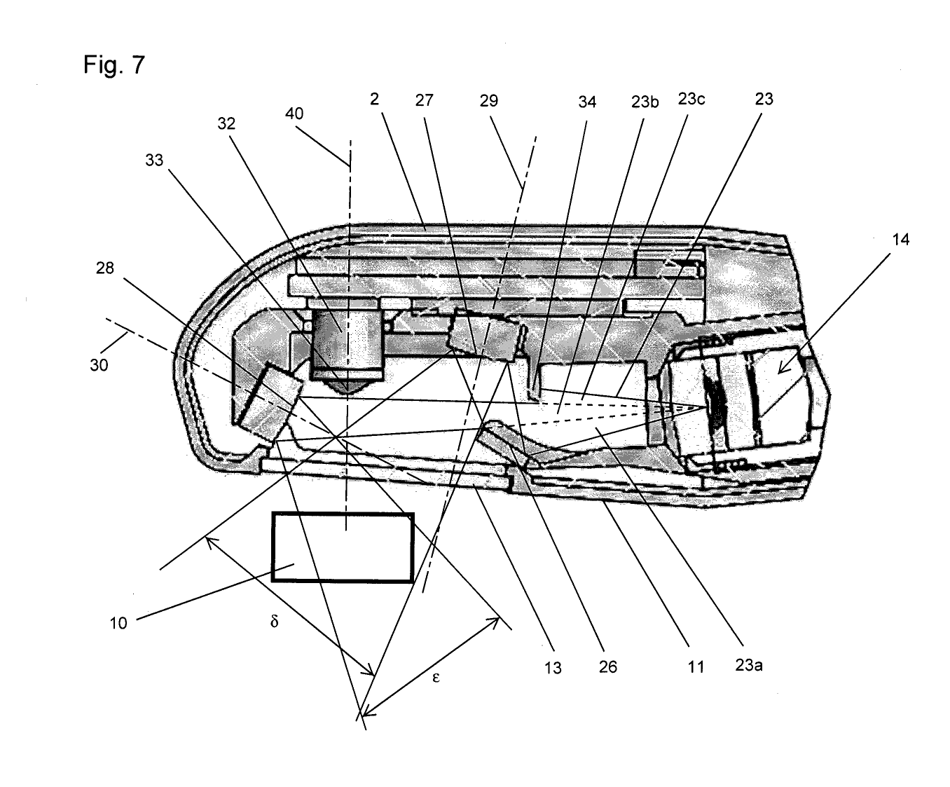

FIG. 7 shows a detail of FIG. 6,

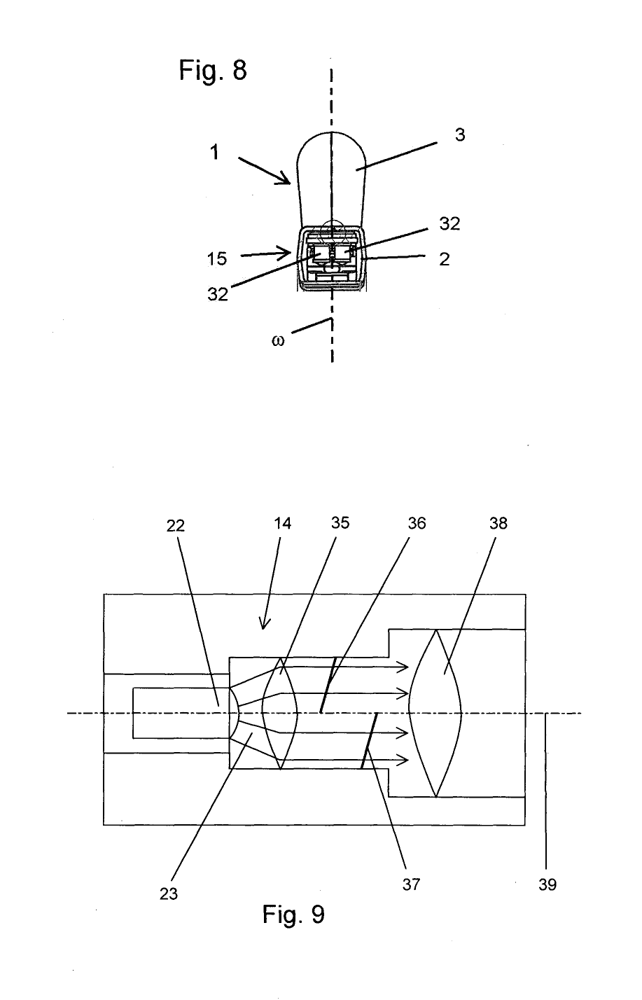

FIG. 8 shows a section through the device along line VIII-VIII, and

FIG. 9 shows a detail of an embodiment of a projector.

In the drawings, a preferred embodiment of a device 1 for three-dimensional imaging of objects 10, in particular teeth, is shown, which has an imaging area 2 and a gripping area 3. Between the imaging area 2 and the gripping area 3, a central area 5 is arranged in the embodiment depicted. Since the central area 5 has a smaller outside dimension than the imaging area 2, the imaging area 2 has an essentially conical transition area 6 to the central area 5. On the front end 4, the imaging area 2 is rounded.

The imaging area 2 has a center axis 7, the gripping area 3 has the center axis 8, and the central area 5 has a center axis 9. The angle α between the center axis 7 and the center axis 8 lies between 10° and 40° according to the invention, whereby this angle α in the embodiment depicted (with a central area 5) is divided into two angles β and γ, whereby the angle β lies between the center axis 7 of the imaging part 2 and the center axis 9 of the central area 5, and the angle γ lies between the center axis 9 of the central area 5 and the center axis 8 of the gripping part 3. The angle β preferably lies between 3° and 15°, and the angle γ lies between 7° and 25°. The length of the imaging area preferably lies between 10 and 60 mm, since within these limits, both an easy handling of the handpiece 1 and sufficient space for installing the projection and/or imaging technology are present.

On its side 11 that faces the object 10 that is to be imaged, an opening 12 (FIG. 5) is arranged in the imaging part 2, which is sealed by a disk 13. Through this disk 13, light, in particular a random pattern, can be directed onto the object 10 with a projector 14, and images of the object 10 can be recorded with a camera system 15. Relative to the gripping area 3, the imaging area 2 is thus tilted backward against the projection direction by an angle α of between 10° and 40°.

In FIGS. 5 to 8, an embodiment of the invention is depicted, in which the projector 14 irradiates a light beam 23 with a light source. The light beam 23 enters through one or more transparent vehicles 36, 37, depicted in FIG. 9, for example slides, on which a pattern, arranged according to a random principle, is arranged. The pattern preferably consists of essentially randomly distributed, optionally irregularly formed points and/or lines, which are subsequently projected onto the object 10, for example a tooth.

In the beam path of the light beam 23, there is a deflection mirror 26, which deflects one part 23a of the light beam 23, the lowermost part in the embodiment of FIG. 7, to a first mirror 27, which subsequently directs the light to the object 10. Another part 23b of the light beam 23, the central part in the embodiment of FIG. 7, strikes a second minor 28 directly, from which the light is also directed to the object 10.

The deflection mirror 26 is preferably a flat mirror, but it could also be a convex or concave minor, if necessary. The two mirrors 27 and 28 are preferably two-axis convex minors with the same or different radii of curvature in the two axes, with which the respective proportion of the ray beam 23 can be more greatly scattered, if necessary.

In the embodiment depicted, the arrangement and the curvature of the deflection minor 26 and the first minor 27 are selected in such a way that the part 23a of the light beam 23 in the image plane of the drawing has an opening angle δ of approximately 30°. By way of example, the arrangement and curvature of the second minor 28 are selected in such a way that the part 23b of the light beam 23 in the image plane of the drawing has an opening angle ε of approximately 25°. The opening angle of parts 23a, 23b of the light beam 23 in a normal direction relative to the image plane of the drawing can be the same or different, depending on the requirement, in the respective opening angles δ, ε lying in the image plane because of the suitable curvature of the mirrors 27, 28.

Because of the arrangement of the mirrors 27, 28 selected by way of example in FIG. 7, the optical axes 29, 30 of said mirrors are tilted toward one another in such a way that the light beam parts 23a, 23b strike the object 10 from different directions.

In the projection direction of the projector 14 viewed between the two mirrors 27, 28, in the embodiment depicted somewhat nearer to the second mirror 28, a camera system 15 is arranged, which in the embodiment depicted consists of two cameras 32, which record stereoscopic images for three-dimensional measurement of the object 10, by images being recorded from different directions with imaging areas that overlap one another. The two optical axes 29, 30 of the mirrors 27, 28 span a plane ω, whereby the two cameras 32, more precisely their lenses 33, are symmetric to both sides of this plane ω.

By this preferred arrangement, the camera system 15 lies with the mirrors 26, 27 or their optical axes 29, 30 in a plane w, which makes possible very precise image recording and thus measuring of the object. By the projection of the light beam parts 23a, 23b by mirrors 26, 27, which lie on both sides of the camera system 15, an illumination or projection of the random pattern onto the object 10 also takes place from two sides in this plane ω, by which—viewed from the standpoint of the camera system 15—shadows or flaws on the object 10, which can occur, for example, in the case of molars or incisors, can be very reliably avoided.

In principle, it would also be possible to position the minors—viewed from the projector 14—in addition to the two cameras 32 and optionally to rotate the two cameras 32 by 90° so that they both lie in the plane ω. More than two mirrors, both in front of and/or behind and lateral to the cameras 32, are also conceivable to produce the best possible illumination or pattern projection on the object 10.

In the embodiment depicted, in the area above the deflection minor 26, an aperture 34 is arranged that blocks a third part 23c of the light beam 23 so that the latter does not cause any undesirable reflections in the optics 33 of the cameras 32. Depending on the arrangement of the minors 26, 27, 28 and the lenses 33, the aperture 34 can also be omitted or arranged or formed differently.

All mirrors 26, 27, 28, the aperture 34, and optionally also the camera system 15 can be fastened in an adjustable manner to corresponding holding devices 31 so that if necessary, a simple adjustment and/or calibration of the individual components is possible. In addition, all or even only a portion of the above-described components can be fastened to a vehicle system and preadjusted, which then can be used in an imaging device. The housing of the device 1 preferably consists of two housing halves 16, 17, which are designed as a mirror image, by which the device can be assembled very easily.

By the selected arrangement according to the invention that is depicted by way of example in the drawing, a very compact and thin design is possible, which can be integrated, for example, very readily in a handpiece for three-dimensional imaging of teeth.

The arrangement of mirrors and cameras described in connection with FIGS. 5 to 8 is preferably used in handpieces with an angled imaging area 2 and optionally central area 5, since an especially good option is offered by the minors 26, 27, 28 to incorporate the entire projection and imaging technology into an angled handpiece 1, which is very thin and especially easily handled owing to the sharp bend in particular in the case of oral scanners.

In the device according to the invention, an optimal degree of sharpness can be achieved directly starting from the outside surface of the scanner glass 13 without the risk of shadows or flaws—just by placing the scanner on an object, e.g., a tooth, the latter can be measured; conversely, known scanners often have to be held at a certain distance from the teeth, which significantly hampers the imaging process, in comparison to the possibility according to the invention of also being able to be placed directly on the teeth.

In FIG. 9, an embodiment of the invention is diagrammatically depicted in which two transparent vehicles 36, 37, for example two slides, on which patterns arranged according to a random principle are arranged, lie in the beam path of a light beam 23, which is irradiated from a light source 22, for example an LED. The pattern can essentially consist of randomly distributed, optionally irregularly formed points and/or lines. The light goes into the embodiment depicted first through a lens 35, then through the two vehicles 36, 37, and subsequently through another lens system that is symbolically depicted by a lens 38 and that is used to orient the projection beam 23 and to adjust sharpness.

The projector 14 of FIG. 9 can be used, for example, in a device that is depicted in FIGS. 5 to 8, in which the light beam 23 is directed via two minors 27 and 28 to an object 10. Since the light traverses paths of different lengths, depending on whether it strikes either via the deflection minor 26 and the mirror 27 or via the minor 28 on the object 10, blurring of one or the other or both projections can develop in the projection of the pattern, present on a vehicle, onto the object 10.

By using two vehicles 36, 37, this can be taken into account, and blurs can be compensated for individually. This can be done, for example, in that the two vehicles 36, 37 are offset with respect to one another in the propagation direction of the light. Thus, e.g., the vehicle 36, which lies in the beam path of the light beam 23b of the mirror 28, is further behind or further away from the lens 38 or a subsequent lens system than the vehicle 37, which lies in the beam path of the light beam 23a of the mirror 27 so that altogether, the path of the light from the respective vehicle 36, 37 via the respective mirror(s) 26, 27, 28 to the object 10 is again approximately the same length. The different distances from the vehicles 36, 37 to the lens system 38 are decisive, since these distances determine the position and location of the definition plane in the measuring space. If the path difference via the respective mirror(s) 26, 27, 28 to the object 10 is not very large, for example, also only a single vehicle could be used, which either has sections offset in stages or which is even, however, correspondingly greatly tilted to compensate for the path difference in the center. As another option, a single vehicle could also be used, which is coated with a pattern on different areas or sections in each case on the front side and on the back side. The thickness of the vehicle material then determines the distance difference.

In FIG. 9, a tilting of the vehicles 36, 37 relative to the propagation direction of the light can be seen, i.e., the vehicles 36, 37 are not exactly at a right angle relative to the propagation direction of the light. This embodiment of the invention is advantageous when the projector 14 or its optical center axis 39, as in the embodiment depicted in FIGS. 1 to 8, is oriented at an angle β that is greater than 0° in the imaging area 2; in particular, the optical center axis 39 is not oriented at a right angle to the optical center axis 40 of the camera 32. The arrangement of the projector 14 in the angled transition area between the imaging area 2, in which the cameras 32 are located, and the central area 5 is especially advantageous, since in this way, the imaging area 2 can be kept relatively short, which significantly improves the handling of the handpiece 1. Blurring of the pattern projected onto the object 10 caused by tilting the projector 14 can be compensated for by tilting the vehicles 36, 37.

1. Device for recording images of three-dimensional objects (10), in particular teeth, with an imaging area (2), in which at least one mirror (26, 27, 28) is arranged for deflecting a light or projection beam (23) and/or an image that is reflected by the object (10), and with a gripping area (3), characterized in that a camera (32) and/or a projector (14) is/are arranged in the imaging area (2) and in that the imaging area (2) is tilted against the projection direction by an angle (α) of between 10° and 40° relative to the gripping area (3).

2. Device according to claim 1, wherein a central area (5) is arranged between the gripping area (3) and the imaging area (2), which central area is tilted in each case by an angle (β, γ) of at least 3° relative to the gripping area (3) and the imaging area (2).

3. Device according to claim 1, wherein the central area (5) is tilted by an angle (γ) of 7° to 25° relative to the gripping area (3).

4. Device according to claim 1, wherein the imaging area (2) is tilted by an angle (β) of 3° to 15° relative to the central area (5).

5. Device according to claim 1, wherein the imaging area (2) has a length of between 10 mm and 60 mm.

6. Device according to claim 1, wherein the imaging area (2) has a housing with an opening (12) that is preferably closed by a transparent disk (13).

7. Device according to claim 1, wherein it has a housing with two mirror-symmetrical housing halves (16, 17).

8. Device according to claim 1, wherein the projector (14) is arranged in the transition area between the imaging area (2) and the adjacent central area (5) or the gripping area (3).

9. Device according to claim 1, wherein at least two mirrors (27, 28) in each case reflect a light beam (23a, 23b) from the projector (14) from different directions onto the object (10).

10. Device according to claim 9, wherein light is directed onto a first mirror (27) indirectly via a deflection mirror (26), and light is directed onto a second mirror (28) directly from the projector (14).

11. Device according to claim 9, wherein two mirrors (27, 28) lie on different sides of the camera (32).

12. Device according to claim 1, characterized by two cameras (32) with imaging areas that overlap one another and that record images from different directions.

13. Device according to claim 1, wherein the projector (14) projects a pattern, arranged according to a random principle, onto the object (10).

14. Device according to claim 13, wherein at least one transparent vehicle (36, 37) is arranged with the pattern in the beam path of the projector (14).

15. Device according to claim 13, wherein the pattern essentially consists of randomly distributed, optionally irregularly formed points and/or lines.

16. Device according to claim 14, wherein the vehicle (36, 37) has sections that are offset with respect to one another in the direction of the beam path.

17. Device according to claim 14, wherein at least two vehicles (36, 37) are offset with respect to one another in the direction of the beam path.

18. Device according to claim 17, wherein at least one vehicle (36, 37) has sections that are offset with respect to one another in the direction of the beam path.

19. Device according to claim 14, wherein with reference to the propagation direction of the light, at least one vehicle (36, 37) is tilted at an angle unequal to 90°.
